(19) United States
(12) Patent Application Publication
(10) Pub. No.: US 2021/0068988 A1
(43) Pub. Date: Mar. 11, 2021

(54) Driving Assembly for Moving Body Part

(71) Applicant: Sze Kit Ho, Hong Kong (HK)

(72) Inventor: Sze Kit Ho, Hong Kong (HK)

(21) Appl. No.: 16/771,683

(22) PCT Filed: Dec. 5, 2018
(86) PCT No.: PCT/IB2018/059676

§ 371 Date: Jun. 11, 2020

(30) Foreign Application Priority Data
Nov. 12, 2017 (AU) 2017904968
Jan. 13, 2018 (AU) 2018900103

(51) Int. Cl. A61F 2/58; A61F 2/72; A61F 5/01

(52) U.S. Cl. CPC A61F 2/586; A61F 2002/6836; A61F 5/013; A61F 2/72

(57) ABSTRACT

A driving assembly for moving a first body part relative to a second body part comprising at least one manipulator unit for body part including a housing for accommodating at least partly of an actuator, a torque/force transmission gear, a partial gear wheel operatively connected with each other and a first connector arranged at a first/tail end of the manipulator unit for coupling with the first body part and a second connector movably arranged at a second/head end of the manipulator unit for coupling with the second body part and driven by the partial gear wheel actuated by the actuator via the torque transmission gear for coupling and moving with the second body part, and preferably an arc channel or rail for guiding back and forth movement of the partial gear wheel.

### TECHNICAL FIELD

The present disclosure relates to a driving assembly, and more particularly to a driving assembly for moving or rotating, or assisting or resisting movement of, a first body part relative to a second body part, whereby enabling flexion, extension, pronation, supination, rotation, abduction, and adduction of the first body part relative to the second body part.

### BACKGROUND ART

Existing technologies for manipulating body parts are usually configured for: positioning the motors/actuators directly on the joint rotation axial; or positioning a mechanical joint directly on the joint rotation axial and have the extension to be manipulated by actuators at a remote/distance site. Both designs require the body joint to be covered by motors and/or mechanical joints on one and/or both sides. Such an over-the-joint design requires the body part to be fitted in between the mechanical parts, and because of different limb circumferences, different sizes are needed. Sometimes the motors/actuators get into the way of the body movement. And most importantly, these prior arts are incapable to manipulate joints that have the axial physically blocked or covered.

Further, each year there are more than 18 millions people suffer from stroke and some of them lost some of the body functions or control over various body parts, such as limbs including fingers, hands, legs and the like, due to the damage of brain neurons. They need to practice the body functions in order to relearn the control thereof. In this regard, a manipulator capable of assisting control and movement for the impaired body parts, such as limbs, is highly desirable in the art.

### BRIEF SUMMARY OF INVENTION

According to one aspect of the present disclosure, there is a driving assembly for moving or rotating, or assisting or resisting movement of, a first body part relative to a second body part and preferably enabling flexion, extension, pronation, supination, rotation, abduction, and adduction of the first body part relative to the second body part, comprising at least one manipulator unit for body part including a housing for accommodating at least partly of an actuator, a torque/force transmission gear, a partial gear wheel operatively connected with each other and a first connector arranged at a first/tail end of the manipulator unit for coupling with the first body part and a second connector movably arranged at a second/head end of the manipulator unit for coupling with the second body part and preferably further coupled with and driven by the partial gear wheel actuated by the actuator via the torque transmission gear for coupling and moving with the second body part, and preferably an arc channel or rail for confining and/or guiding reciprocal or back and forth movement and preferably rotational movement of the partial gear wheel; wherein the second connector is configured to be operated interchangeably between an idle/inactive state in which the second connector is positioned at or adjacent to the second end and an active state in which the second connector is driven to move away from or move toward the second end along a circumferential direction by the partial gear wheel or to rotate a predetermined or desired first angle about a rotation axis defined by a curvature of the partial gear wheel and preferably the arc channel or rail in response to a preset instruction or a received real-time instruction, so as to drive the second body part coupled therewith to rotate/pivot a predetermined or desired second angle about a body joint positioned between the first and the second body parts.

According to a further aspect of the present disclosure, there is a driving assembly for moving or rotating, or assisting or resisting movement of, a first body part relative to a second body part and preferably enabling flexion, extension, pronation, supination, rotation, abduction, and adduction of the first body part relative to the second body part, comprising at least one manipulator unit for body part including a first connector arranged at a first/tail end of the manipulator unit for coupling with the first body part and a second connector movably arranged at a second/head end of the manipulator unit for coupling and moving with the second body part, and a curved driving member positioned between the first end and the second end, wherein the second connector is actuated and driven by the curved driving member for effecting reciprocal or back and forth movement and preferably rotational movement along a curved path between the first connector and the second connector; wherein the second connector is configured to be operated interchangeably between an idle/inactive state in which the second connector is positioned at or adjacent to the second end and an active state in which the second connector is driven to move away from or move toward the second end along a circumferential direction by the curved driving member or to rotate a predetermined or desired first angle about a first rotation axis defined by a curvature of the curved driving member in response to a preset instruction or a received real-time instruction, so as to drive the second body part coupled therewith to rotate/pivot a predetermined or desired second angle about a body joint/a second rotation axis positioned between the first and the second body parts; and preferably, the first rotation axis is substantially parallel to or coinciding with the second rotation axis for optimizing a driving efficiency thereof. In some embodiments, the curved driving member comprises a housing for accommodating at least partly of an actuator, a torque/force transmission gear, a partial gear wheel operatively connected with each other; wherein the second connector is connected with and driven by the partial gear wheel actuated by the actuator via the torque transmission gear, so as to be moved interchangeably between a first angular position and a second angular position along the circumferential direction in response to the preset instruction or the received real-time instruction.

In some embodiments, the driving assembly comprises a plurality of manipulator units of identical dimension or of different dimensions arranged in serial and/or parallel connection with each other; wherein respective first connectors of the plurality of manipulator units are configured to couple with each other and/or with same first body part and respective second connectors of the plurality of manipulator units are configured to couple with each other and/or with same second body part for providing extra degrees of freedom of rotation or motion for the second body part in relation to the first body part.

In other embodiments, the second connector of one of the plurality of manipulator units is configured to couple with the first connector of other one of the plurality of manipulator units, so as to provide additional rotation angle and/or extra degrees of freedom of motion for one or more body parts or joints coupled with and driven singly/respectively or collectively by one or more of the plurality of manipulator units.

In further embodiments, the driving assembly further comprises one or more stoppers arranged at, and preferably adjacent to a tail end of, the partial gear wheel, the arc channel or rail, and/or the housing for confining movement and/or providing safety limits for movement of the partial gear wheel operated in both idle and active states.

In yet still other embodiments, the actuator is selected from a group comprising a DC motor, an AC/DC motor, a hydraulic motor, a pneumatic motor, a brush/brushless motor, a piezo motor, a memory shaped alloy actuated by AC/DC electricity, batteries, chemicals, change of temperatures, change of pressures, lights or electromagnetism, sound or energy waves; and/or wherein the torque transmission gear is selected from a group comprising a worm gear, a spur gear, a bevel gear, a belt gear, a pulley gear, and a combination thereof; and/or wherein the partial gear wheel is selected from a group comprising a worm wheel, a spur wheel, a bevel wheel, a belt wheel, a pulley wheel, and a combination thereof; and/or wherein the partial gear wheel is a wheel with less than 360 degrees, preferably less than 135 degrees, but more than 0 degree of rotation.

In several embodiments, the manipulator unit further comprises one or more sensors and/or one or more controllers arranged at or in the housing to measure or control/process data related to positions, angles of rotation, degrees of movement, or orientations of the second connector, forces or pressures exerted or received by the second connector, ambient sound, light, and/or temperature; wherein measured and output or feedback data generated by the sensors and/or the controllers are communicated or transmitted, preferably to a remote/external terminal, via one or more of wired and/or wireless technologies and protocols including Bluetooth, Wifi, Infrared, Zigbee, GSM, LTE, and a combination thereof.

In other embodiments, an adjacent pair of a first and a second manipulator units are coupled with each other via an interconnection of the second connector of the first manipulator unit and the first connector of the second manipulator unit, an interconnection of the first connector of the first manipulator unit and the second connector of the second manipulator unit, an interconnection of the first connector of the first manipulator unit and the first connector of the second manipulator unit, or an interconnection of the second connector of the first manipulator unit and the second connector of the second manipulator unit.

According to another aspect of the present disclosure, it discloses a robotic hand or glove for moving or rotating, or assisting or resisting movement of, a human hand and/or its fingers comprising a foregoing driving assembly, further comprising a palm plate for mounting the robotic hand or glove on the human hand wrist, and/or a forearm and for mounting a plurality of manipulator units for use with and manipulation of a plurality of fingers of the human hand, wherein each of the plurality fingers is coupled with and actuated by one or more manipulator units configured for and capable of providing one or more degrees of freedom; and one or more sensors or sensing devices including an electromyography sensor for measuring electrical activity of muscles of the human hand and fingers, a force sensitive resistor for measuring or gathering information about pressure at each fingertip of the human hand, an accelerometer for measuring acceleration forces of the human hand and fingers, and/or a gyroscope for measuring or maintaining orientation and angular velocity of the human hand and fingers; a micro-controller configured to control/manipulate, coordinate, and/or synchronize movement of the plurality of manipulator units in response to data measured and provided by the one or more sensors for effecting desired operations of the robotic hand for assisting or resisting movement of the human hand.

In some other embodiments, the palm plate further comprises at least one first attachment connector having a first engagement member arranged at or in proximity of a proximal end of the palm plate for coupling with a human wrist and/or forearm and/or at least one second attachment connector having a second engagement member arranged at or in proximity of a distal end of the palm plate for coupling with the first connector of a proximal one of the plurality of manipulator units.

In further embodiments, two manipulator units arranged in a cascaded manner are coupling with one of the fingers or thumb of the human hand to provide 2 degrees of freedom and actuate rotation of one or more finger joints including proximal interphalangeal (PIP) joint and/or metacarpophalangeal (MCP) joint to provide flexion and/or extension of the finger or thumb; and/or two or three manipulator units arranged in a cascaded manner are coupling with other one of the fingers, including index finger, middle finger, ring finger, and little finger, of the human hand to provide 2 or 3 degrees of freedom and actuate rotation of one or more finger joints including PIP joint, MCP joint, and/or distal interphalangeal (DIP) joint; or thumb of the human hand to provide 2 or 3 degrees of freedom and actuate rotation of one or more finger joints including interphalangeal (IP) joint, MCP joint, and/or carpal bone and metacarpal (CMC) joint; and/or wherein extra one or more manipulators arranged in standalone or combination with others are coupling with the thumb and other fingers to provide extra degree of freedom for abduction and/or adduction.

In some embodiments, each of manipulator units is configured to provide itself or a segment of a finger of the human hand with rotation movement preferably greater than 0 degree and more preferably less than 180 degree.

### BRIEF DESCRIPTION OF DRAWINGS

The present disclosure will be described in details below with reference to the accompanying drawings, in which:

FIGS. 1 and 1a are respectively the perspective and side view of an example driving assembly adapted for moving an associated component/object according to a preferred embodiment of the present disclosure;

FIGS. 2a-2d are respectively the partially cutaway view and perspective views of another example driving assembly adapted for moving an associated component/object according to a preferred embodiment of the present disclosure;

FIGS. 3a-3g are respectively the perspective views of other example driving assembly adapted for moving an associated component/object according to preferred embodiments of the present disclosure;

FIGS. 4a-4l are respectively the perspective views of other example driving assembly adapted for moving associated body parts according to preferred embodiments of the present disclosure;

FIGS. 5a-5c are respectively the perspective view and top view of another example driving assembly/glove adapted for moving associated body parts according to preferred embodiments of the present disclosure; and

FIG. 6 is a perspective side view of a further example driving assembly for moving associated body parts according to a preferred embodiment of the present disclosure.

### DETAILED DESCRIPTION OF INVENTION

The present disclosure will now be described in further details with reference to the accompanying drawings and embodiments, so as to make the objects, technical solutions and advantages of the present disclosure more apparent.

According to the present disclosure, it provides a body joint manipulator devised to rotate body parts according to the joint axial, and allows the manipulation of body parts in which the body joints are inaccessible. The manipulator consists of an integration of an actuator, a transmission gear, a partial gear wheel as a moving arm and a housing preferably having an arc channel to guide the movement of the geared arm. The geared arm and/or arc channel have connectors attached to the body parts and/or connect to each other. The manipulator can be configured as a single joint manipulator or it could be cascaded for multiple joints manipulation or composite movement manipulation of a single joint.

In some embodiments, the technical solution of the present disclosure is able to manipulate a body joint without covering the joint axial, it is done by confining the movement within the arc channel where the rotation centre at the joint axial. Thus it is easy to mount and demount the manipulators on and from the body parts without requiring any fitting. For joints that are blocked by other body parts, the technical solution can manipulate the movement of the joints at ease. Another advantage of the technical solution is its cascadability. Multiple manipulators can be cascaded together through connectors of different lengths. Thus it is able to cover the whole body limb in one configuration.

According to the present disclosure, it provides a body joint manipulator which is capable to move or rotate body parts along the corresponding body joints without occupying the space at the joint axial. This manipulator constitutes of an actuator, a torque transmission gear, a partial gear wheel, a housing for the mechanical parts (actuator, the gear and partial gear wheel) combined with an arc channel or rail to guide the movement of the partial gear wheel (to be moved out or in the arc of the housing) and connectors at the tail of the housing and the head/tip of the geared wheel to allow fixture on the body parts as well as other mechanical parts (including cascade on another adjacent manipulator), where the actuator is a DC motor but can also be other actuators for example AC/DC motors, hydraulic, pneumatics, brush/brushless, or the like.

In some embodiments, the gear might be a worm gear but can also be other gears for example spur gear, bevel gear, belt gear, pulley gear, or the like, with one or more combination of multiple gears or belts or cables; wherein the partial gear wheel is a worm wheel but can also be other gear wheel for example spur wheel, bevel wheel, belt wheel, pulley wheel, or the like; and wherein the partial gear wheel is a wheel with less than 360 degrees but more than 0 degree of rotation.

In other embodiments, the housing has an arc channel or rail with less than 360 degrees but more than 0 degree to allow rotation movement of the gear wheel with less than 360 degrees but more than 0 degree, wherein the movement of the partial gear wheel is confined by the arc channel of the housing and/or one or more rails on the side of the housing using but not limited to rods, bearings, rollers, or the like; and preferably the rails and/or the arc channel of the housing has one or more mechanical stoppers to confine the forward or backward movement of the gear wheel (move in or out of the housing).

In further embodiments, the housing has internal structure to confine the location of the motor/actuator, gear and gear wheel in order to maintain the center distances between the gear or gear wheel; wherein the housing can be one complete part or a combination of different parts joined together using but not limited to screws, nails, ultrasound, or the like; and/or wherein the gear, gear wheel and housing can be of different materials such as but not limited to plastics, metals, composite materials, or the like; and/or wherein the gear, gear wheel and housing can be manufactured by different methods such as but not limited to CNC, drilling and milling, casting, injection molding, 3D printing, stamping, or the like.

In several example embodiments, wherein the connector can be located at any part of the housing and can be connected to any part of the manipulator of another copy and/or any part of the body; and/or wherein the connector can be of any length and of any shape to fit another manipulator and/or different body parts of different size.

According to the present disclosure, the manipulator can be applied on different body joints (including but not limited to) such as shoulder, elbow, wrist, finger, hip, knee and ankle, or the like; and/or the manipulator can assist or resist movements (including but not limited to) such as flexion, extension, abduction, adduction, rotation, grasp, open/close, or the like; and/or wherein the manipulator can be connected with another manipulator to provide additional rotation angle and/or more degree of freedom for one or more body joints; and/or wherein the manipulator can be cascaded together with one or more copies of this manipulator of different actuators, of different gears, of different sizes, of different range of motions, of different materials, of different shapes, of different connectors, of different fixtures, or the like; and/or wherein the manipulator can be connected to another manipulator with different arrangement (including but not limited to) such as end to tip, tip to tip, end to end, or the like; and/or wherein the manipulator can be arranged with one or more manipulators arranged in serial or parallel, in single or in multiple according to different body parts or joints (including but not limited to) such as a shoulder joint capable of flexion/extension and abduction/adduction and rotation, a wrist joint with capabilities of flexion/extension and abduction/adduction, pronation/supination and rotation, a hand with multiple fingers joints with capabilities of flexion/extension and abduction/adduction, a hip joint with capabilities of flexion/extension and abduction/adduction and rotation, an ankle joint with capabilities of flexion/extension and abduction/adduction and rotation, and other applicable body joints or the like.

According to the present disclosure, it further provides an exoskeleton robotic hand design having a body part manipulator or remote center manipulator (RCM), comprising a worm gear, a partial worm wheel, and preferably an arc rail channel to guide a remote center (RC) movement for actuating fingers of a human hand. The robotic hand consists of more than 1 fingers with dual RCM for each finger to manipulate two finger joints—the PIP joint and MCP joint. The same manipulator can also actuate DIP and wrist joint if needed. This robotic hand assists people with weak muscle strength and poor muscle coordination of hand functions to carry out normal hand functions with better strength and faster speed.

The RCM Robotic Hand is designed to actuate human hand fingers to assist the movement and enhance the strength. It consists of at least one manipulator for each finger. Each manipulator can actuate the finger movement according to its center of rotation. The RCM Robotic Hand can provide 10 degrees of freedom for 5 fingers manipulation. Each manipulator can provide greater than 90 degrees of freedom of rotation or range of motion. The RCM Robotic Hand is configured to provide assistive hand functions to help people to use their hand even though they have poor muscle strength and/or coordination.

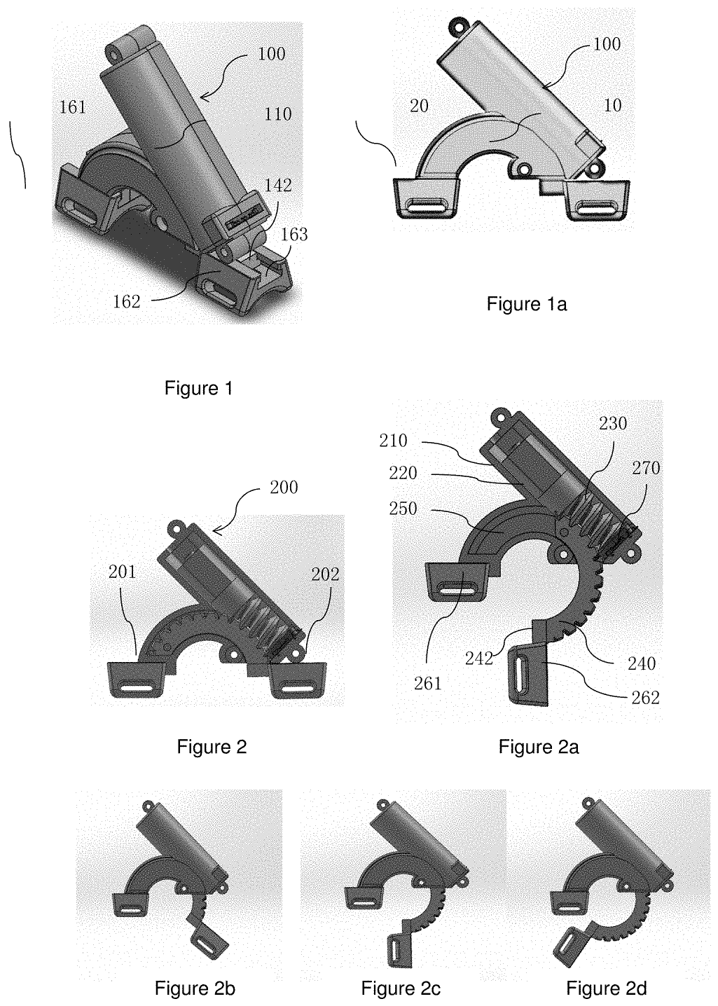

Referring to FIGS. 1 and 1a, a perspective and a side view of an example driving assembly adapted for controlling an associated component/object according to a preferred embodiment of the present disclosure are illustrated, according to which the driving assembly comprises a manipulator unit 100 for moving a first part relative to a second part including a housing 110 for accommodating at least partly of an actuator, a torque/force transmission gear, a partial gear wheel operatively connected with each other and a first connector 161 arranged at a first/tail end of the manipulator unit for coupling with the first part and a second connector 162 movably arranged at and coupled with a second/head end of the manipulator unit via its coupling mechanism 163 for coupling with the second part and preferably further coupled with and driven by a free end 142 of the partial gear wheel actuated by the actuator via the torque transmission gear for coupling and moving with the second part. The partial gear wheel will be completely received and concealed in the housing while in its idle state. It will be extending out or retracting into the housing via an opening arranged at the housing and preferably positioned at the second end of the manipulator unit while in its active state. As illustrated in FIG. 1a, the manipulator 100 might comprise a first portion 10 adapted for accommodating the actuator and the torque/force transmission gear and a second portion 20 having a part of a substantially semi-circular or curved cross-section for accommodating the partial gear wheel with a curved portion. In some embodiment, the first portion 10 is substantially in the form of a cylindrical casing and the second portion 20 is substantially in the form of a semi-circular or a curved casing for confining and guiding the motion of the partial gear wheel, wherein the cylindrical casing communicates with the curved casing for enabling a proper engagement between the partial gear wheel and the transmission gear.

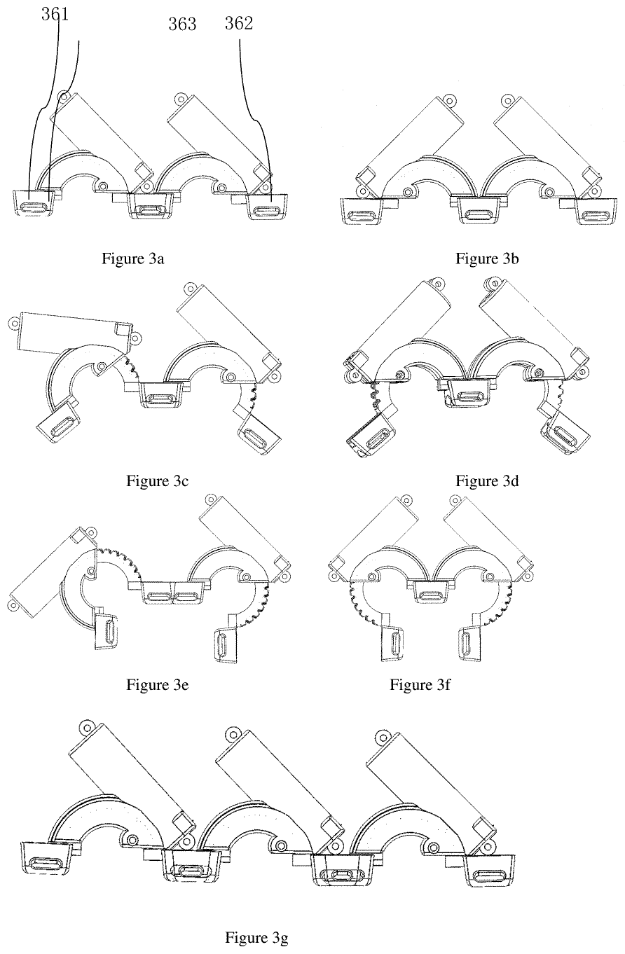

Now referring to FIGS. 2a-2d, partially cutaway view and perspective views of another example driving assembly adapted for moving an associated component/object according to a preferred embodiment of the present disclosure are illustrated. According to the present disclosure, the example driving assembly is adapted for moving or rotating, or assisting or resisting movement, or a first body part relative to a second body part (such as parts of a human/body joint) and preferably enabling flexion, extension, pronation, supination, rotation, abduction, and adduction of the first body part relative to the second body part, wherein the body joint includes but not limited to joints found in fingers, wrists, elbows, shoulders, knees, ankles, toes, neck, hips and human spine. The example driving assembly shown in FIGS. 2-2a comprises at least one manipulator unit 200 for body part including a housing 210 for accommodating at least partly of an actuator 220, a torque/force transmission gear 230, a partial gear wheel 240 operatively connected with each other and a first connector 261 arranged at a first/tail end 201 of the manipulator unit for coupling with the first body part and a second connector 262 movably arranged at a second/head end 202 of the manipulator unit for coupling with the second body part and preferably further coupled with and driven by a free end 242 of the partial gear wheel 240 actuated by the actuator via the torque transmission gear 230 for coupling and moving with the second body part, and preferably an arc channel or rail 250 for confining and/or guiding reciprocal or back and forth movement and preferably rotational movement of the partial gear wheel 240.

In several preferred embodiments, the actuator is selected from a group comprising a DC motor, an AC/DC motor, a hydraulic motor, a pneumatic motor, a brush/brushless motor, a piezo motor, a memory shaped alloy actuated by AC/DC electricity, batteries, chemicals, change of temperatures, change of pressures, lights or electromagnetism, sound or energy waves; and/or wherein the torque transmission gear is selected from a group comprising a worm gear, a spur gear, a bevel gear, a belt gear, a pulley gear, and a combination thereof; and/or wherein the partial gear wheel is selected from a group comprising a worm wheel, a spur wheel, a bevel wheel, a belt wheel, a pulley wheel, and a combination thereof; and/or wherein the partial gear wheel is a wheel with less than 360 degrees but more than 0 degree of rotation.

In some example embodiments, the free end 242 might be in the form of a profiled block with an inverted T-shape cross-section configured to be engageable with a counterpart of the coupling mechanism (such as a groove of a matched cross-section, resembled to and/or denoted by numeral 163 in FIG. 1) of the second connector via tight fit and they could preferably further engaged with each other via a conventional screwing mechanism or coupling mechanism (not shown) well known in the art.

In several embodiments, the manipulator unit further comprises one or more stoppers arranged at, and preferably adjacent to a tail end of, the partial gear wheel, the arc channel or rail, and/or the housing, for confining movement and/or providing safety limits for movement of the partial gear wheel operated in both idle and active states.

In some of further embodiments, the manipulator unit further comprises a sensor 270 configured to measure and feedback a rotation angle or load force of the torque/force transmission gear 230 and/or the partial gear wheel 240 to enable an automatic adjustment of motion of associated body parts controlled by the driving assembly.

In other embodiments, the manipulator unit further comprises one or more sensors and/or one or more controllers arranged at or in the housing to measure or control/process data related to positions, angles of rotation, degrees of movement, or orientations of the free end and/or the second connector, forces or pressures exerted or received by the free end and/or the second connector, ambient sound, light, and/or temperature. In the embodiments, measured and output or feedback data generated by the sensors and/or the controllers are communicated or transmitted, preferably to a remote/external terminal, via one or more of wired and/or wireless technologies and protocols including Bluetooth, Wifi, Infrared, Zigbee, GSM, LTE, and a combination thereof.

According to the present disclosure, the second connector 262 is configured to be operated interchangeably between an idle/inactive state in which the second connector is positioned at or adjacent to the second end 202 and an active state in which the second connector is driven to move away from or move toward the second end 202 preferably along a circumferential direction by the partial gear wheel 240 or to rotate a predetermined or desired first angle about a first rotation axis defined by a curvature of the partial gear wheel and preferably the arc channel or rail in response to a preset instruction or a received real-time instruction, so as to drive the second body part coupled therewith to rotate/pivot a predetermined or desired second angle about a body joint/second rotation axis (not shown) positioned between the first and the second body parts. Preferably, the first rotation axis is substantially parallel to the second rotation axis. In some embodiments, the first rotation axis is substantially coinciding with the second rotation axis for enhancing or optimizing the driving efficiency thereof.

As can be seen from FIGS. 2b-2d, the free end 242 of the partial gear wheel 240 or the second connector 262 is driven to move away from the second end 202 along the circumferential direction confined by the partial gear wheel 240 and/or preferably the arc channel or rail 250 to rotate respectively 45, 90, and 135 degrees of angle about the rotation axis defined by a curvature of the partial gear wheel and/or preferably the arc channel or rail in response to a preset instruction or a received real-time instruction provided by the driving assembly, wherein nearly one fourth, a half, and the whole of the partial gear wheel 240 has been driven and extended out from the housing 210 or the arc channel 250, such that the associated body parts will be driven accordingly to perform a desired motion.

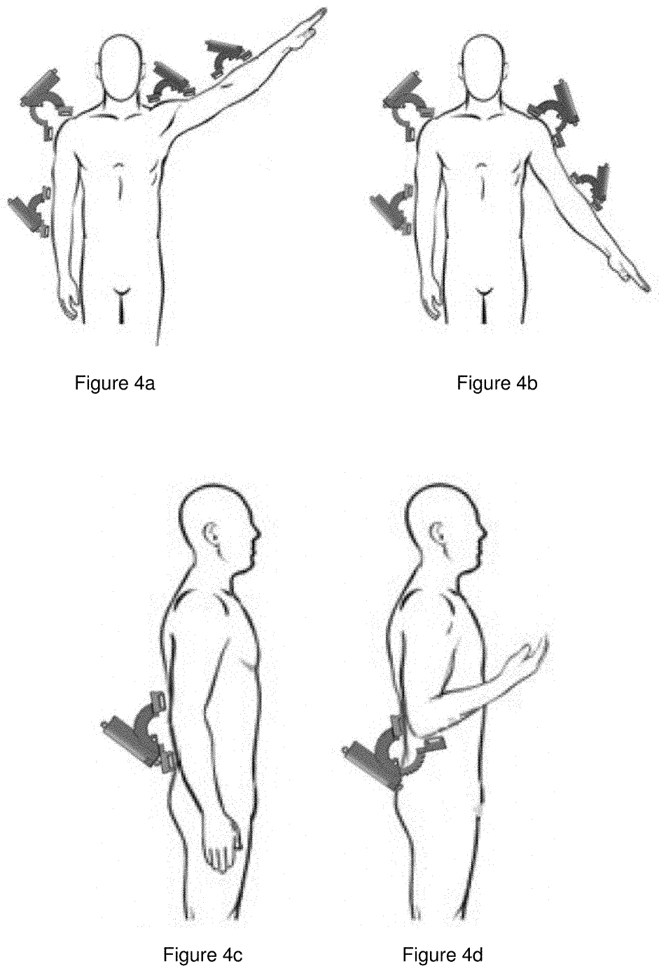

Now referring to FIGS. 3a-3g, the perspective views of other example driving assembly according to preferred embodiments of the present disclosure are illustrated. According to the present disclosure, a plurality of manipulator units of identical dimension or of different dimensions might be arranged in serial and/or parallel connection with each other; wherein respective first connectors of the plurality of manipulator units are configured to couple with each other and/or with same first body part and respective second connectors of the plurality of manipulator units are configured to couple with each other and/or with same second body part for providing extra degrees of freedom of rotation or motion for the second body part in relation to the first body part.

In some embodiments, the second connector of one of the plurality of manipulator units is configured to couple with the first connector of other one of the plurality of manipulator units, so as to provide additional rotation angle and/or extra degrees of freedom of motion for one or more body parts or joints coupled with and driven singly/respectively or collectively by one or more of the plurality of manipulator units.

In yet still other embodiments, an adjacent pair of a first and a second manipulator units are coupled with each other via an interconnection of the second connector of the first manipulator unit and the first connector of the second manipulator unit, an interconnection of the first connector of the first manipulator unit and the second connector of the second manipulator unit, an interconnection of the first connector of the first manipulator unit and the first connector of the second manipulator unit, or an interconnection of the second connector of the first manipulator unit and the second connector of the second manipulator unit.

FIG. 3a has illustrated a head to tail or first configuration having two cascaded manipulators formed by connecting the head end of one of the manipulator units to the tail end of another manipulator unit via a first connector or a second connector acting as an intermediate connector 363, such that two manipulators will equip with only three connectors 361, 362, 363 in total; and FIG. 3b has illustrated a tail to tail or second configuration having two cascaded manipulators formed by connecting together the tail ends thereof; wherein the respective free ends of the partial gear wheels are both in an idle/inactive position and are driven to rotate along a circumferential direction confined by the partial gear wheel by 0 degree of angle.

FIG. 3c has illustrated the first configuration shown in FIG. 3a; and FIG. 3d has illustrated the second configuration shown in FIG. 3b; wherein each of free ends of the partial gear wheels is in an active position and is driven to move along a circumferential direction confined by the partial gear wheel and rotate by 45 degrees of angle, and thus the connector 362 positioned at the right hand side is rotated by 90 degrees of angle in total with respect to the connector 361 positioned at the left hand side.

FIG. 3e has illustrated the first configuration shown in FIG. 3a; and FIG. 3f has illustrated the second configuration shown in FIG. 3b; wherein each of free ends of the partial gear wheels is in an active position and is driven to move along a circumferential direction confined by the partial gear wheel and rotate by 90 degrees of angle, and thus the connector 362 positioned at the right hand side is rotated by 180 degrees of angle in total with respect to the connector 361 positioned at the left hand side. Further, the intermediate connector in FIG. 3e is formed by interconnecting two connectors, namely the second connector of the left manipulator unit and the first connector of the right manipulator unit; wherein these two connectors can be also connected via an intermediate connection member such as a connection rod (not shown) to lengthen the distance between the left manipulator unit and the right manipulator unit.

In some embodiments, three or more manipulators could be cascaded in series or parallel connection with each other, such as another head to tail configuration (or third configuration) as shown in FIG. 3g, which might be adapted to a specific application/joint with many articulation surfaces.

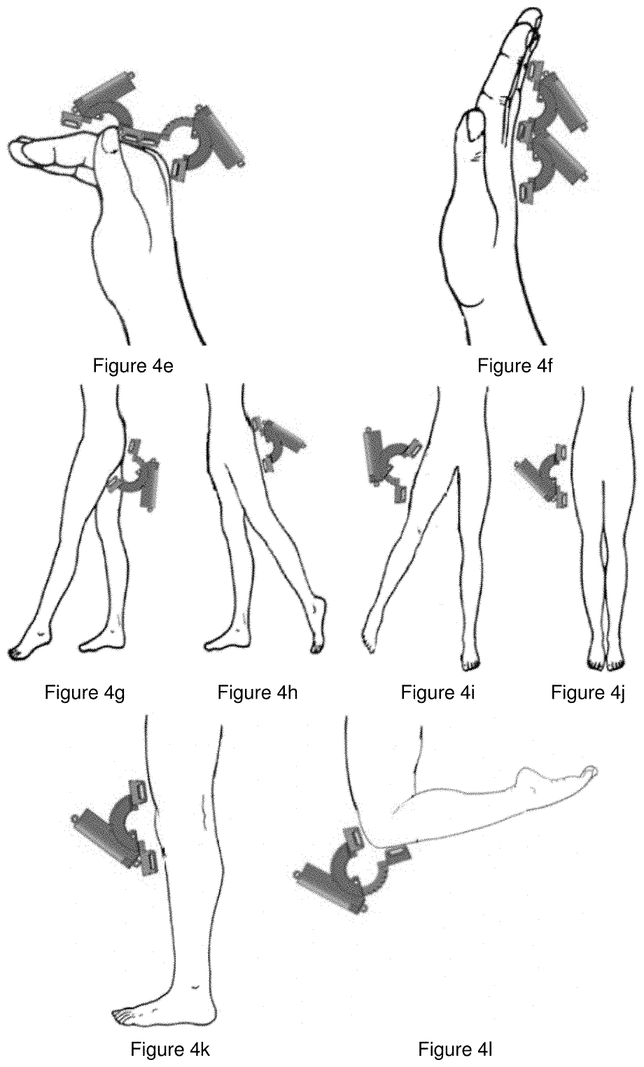

FIGS. 4a-4l are respectively the perspective views of other example driving assembly adapted for moving associated body parts according to preferred embodiments of the present disclosure. As shown in FIGS. 4a-4b, four manipulator units are configured to be positioned in an upper limb covering elbow and shoulder of both arms. In this embodiment, the movement or abduction and adduction of the left arm are controlled by the manipulator unit positioned on the left shoulder.

Referring to FIGS. 4c-4d, one manipulator unit is positioned at the back of the right arm elbow, wherein the first connector arranged at the first end of the manipulator unit is connected with the upper arm and the movable second connector arranged at the second end of the manipulator unit is connected with the forearm, such that various movement, namely the flexion and extension, of the right elbow are driven and controlled by the respective operations of the manipulator unit.

Now referring to FIGS. 4e-4f, two manipulator units in series connection or head to tail configuration are positioned at the back of the hand/palm and a finger, wherein the first connector arranged at the first end of the right manipulator unit is connected with the hand/palm and the movable second connector arranged at the second end of the left manipulator unit is connected with the finger, such that various movement, such as the flexion and extension, of the PIP and MCP joints of the finger are controllable by the respective operations of the left and/or the right manipulator units.

In some embodiments as shown in FIGS. 4g-4j, one or more manipulator units might be positioned at the back or lateral side of the hip joint, wherein the first connector arranged at the first end of the manipulator unit might be connected with the buttock and the movable second connector arranged at the second end of the manipulator unit might be connected with the upper leg, such that various movement, namely the abduction/adduction, flexion/extension, of the hip joint are driven and controlled by the respective operations of the one or more manipulator units.

In some embodiments as illustrated in FIGS. 4k-4l, a manipulator unit is positioned at the knee joint, wherein the first connector arranged at the first end of the manipulator unit is connected with the upper leg and the movable second connector arranged at the second end of the manipulator unit is connected with the lower leg, such that various movement, namely the flexion and extension, of the knee joint are driven and controlled by the respective operations of the manipulator unit.

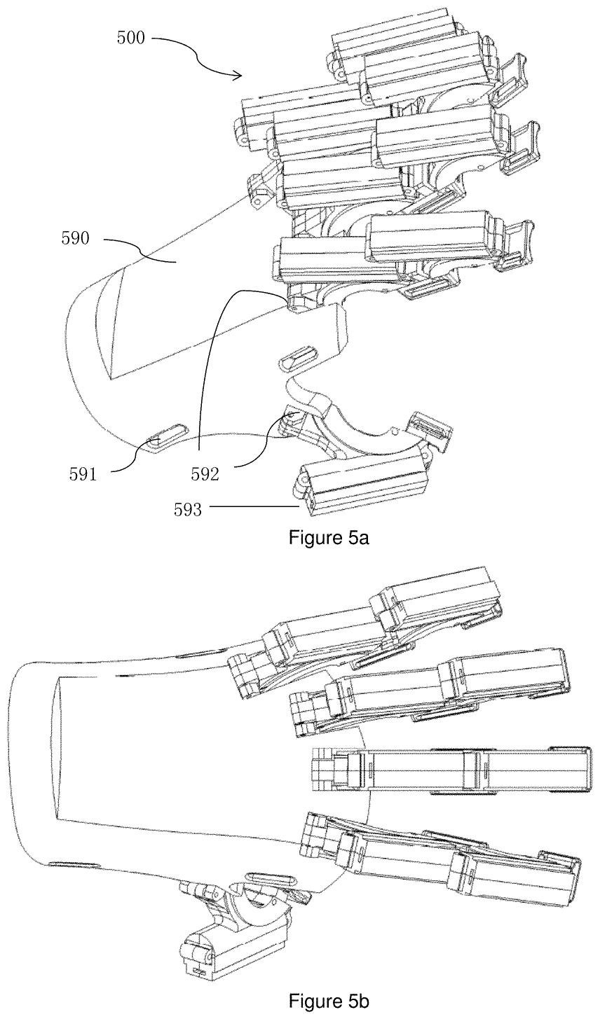

Now referring to FIGS. 5a-5b, the perspective view and top view of another example driving assembly in the form of a robotic hand or glove adapted for moving associated hand and fingers according to preferred embodiments of the present disclosure are shown. The robotic hand or glove 500 is adapted for moving or rotating, or assisting or resisting movement of, a human hand and/or its fingers comprising a foregoing driving assembly, and further comprising a palm plate for mounting the robotic hand or glove on the human hand wrist, and/or a forearm and for mounting a plurality of manipulator units 593 for use with and manipulation of a plurality of fingers of the human hand, wherein each of the plurality fingers is coupled with and actuated by one or more cascaded manipulator units configured for and capable of providing one or more degrees of freedom of motion.

Referring to FIG. 5c, the manipulator unit 593 is similar to the one depicted in FIG. 2a and comprises a housing 510 for accommodating at least partly of an actuator 520, a torque/force transmission gear 530, a partial gear wheel 540 operatively connected with each other and a first connector 561 arranged at a first/tail end of the manipulator unit for coupling with the first body part and/or the palm plate 590 and a second connector 562 movably arranged at a second/head end of the manipulator unit for coupling with the second body part and/or an adjacent manipulator unit and preferably further coupled with and driven by a free end 542 of the partial gear wheel 540 actuated by the actuator via the torque transmission gear 530 for coupling and moving with the second body part, and preferably an arc channel or rail 550 for confining and/or facilitating the guiding of reciprocal or back and forth movement and preferably rotational movement of the partial gear wheel 540.

The housing might be engaged with the palm plate via its coupling mechanism 511, which might be a conventional screwing or fastening mechanism.

In several preferred embodiments, the robotic hand or glove further comprises one or more sensors or sensing devices including an electromyography sensor for measuring electrical activity of muscles of the human hand and fingers, a force sensitive resistor for measuring or gathering information about pressure at each fingertip of the human hand, an accelerometer for measuring acceleration forces of the human hand and fingers, and/or a gyroscope for measuring or maintaining orientation and angular velocity of the human hand and fingers.

In other embodiments, the robotic hand or glove further comprises a micro-controller configured to control/manipulate, coordinate, and/or synchronize movement of the plurality of manipulator units in response to data measured and provided by the one or more sensors for effecting desired operations of the robotic hand for assisting or resisting movement of the human hand.

In yet still other embodiments, the palm plate further comprises at least one first attachment connector 591 having a first engagement member arranged at or in proximity of a proximal end of the palm plate for coupling with a human wrist and/or forearm and/or at least one second attachment connector 592 having a second engagement member arranged at or in proximity of a distal end of the palm plate for coupling with the first connector of a proximal one of the plurality of manipulator units.

In further example embodiments, two manipulator units arranged in a cascaded manner are coupling with one of the fingers or thumb of the human hand to provide 2 degrees of freedom and actuate rotation of one or more finger joints including PIP joint and/or MCP joint to provide flexion and/or extension of the finger or thumb; and/or two or three manipulator units arranged in a cascaded manner are coupling with other one of the fingers, including index finger, middle finger, ring finger, and little finger, of the human hand to provide 2 or 3 degrees of freedom and actuate rotation of one or more finger joints including PIP joint, MCP joint, and/or DIP joint; or thumb of the human hand to provide 2 or 3 degrees of freedom and actuate rotation of one or more finger joints including IP joint, MCP joint, and/or CMC joint; and/or wherein extra one or more manipulators arranged in standalone or combination with others are coupling with the thumb and other fingers to provide extra degree of freedom for abduction and/or adduction.

In several embodiments, each of manipulator units of the robotic hand or glove is configured to provide itself or a segment of a finger of the human hand with rotation movement preferably greater than 0 degree and more preferably less than 180 degree.

According to the present disclosure, the robotic hand or glove 500 can provide more than 2 degree of freedoms for each finger. It employs one or more manipulator/manipulator units to actuate one or more fingers, wherein each finger has more than 1 manipulator to actuate the rotation of the finger joint such as PIP joint and/or MCP joint. Each manipulator is configured to manipulate the finger and to conform/perform the rotation remotely at finger joints, wherein the degrees of rotation (range of motion) and/or the finger joint torque generate by a human being are measured with sensors embedded therein. The arc channel or rail is configured to facilitate the guiding of the movement of the gear wheel and the manipulator further comprises a limited stopper to provide safety limits for the motion of the gear wheel. Two or more manipulators can be connected with each other in different configurations or arrangements such as head to head, tail to tail, head to tail or tail to head as previously illustrated. Each manipulator might equip with a connector at the end of gear wheel and the tail of the housing in order to attach to fingers to be manipulated. The palm plate is configured to secure the manipulators of different fingers and it could be securely attached to the back of a human hand.

As described above, the robotic hand or glove 500 may have attachment connectors (such as 591 depicted in FIG. 5a) for attaching to different fingers and wrist in order to manipulator these body parts. The connector of a manipulator can be attached to a human body or connected to another manipulator. The robotic hand or glove might consist of finger pad for securing the finger on the manipulator, and micro-controller to manipulate the movement of these manipulators; and other sensors such as Electromyography signals sensors, accelerometer and gyroscope, FSR, or the like; and it might be connected wirelessly to other terminals for control and data transfer. In some embodiments, each manipulator can be configured to provide rotation movement for greater than 90 degree but less than 180 degree.

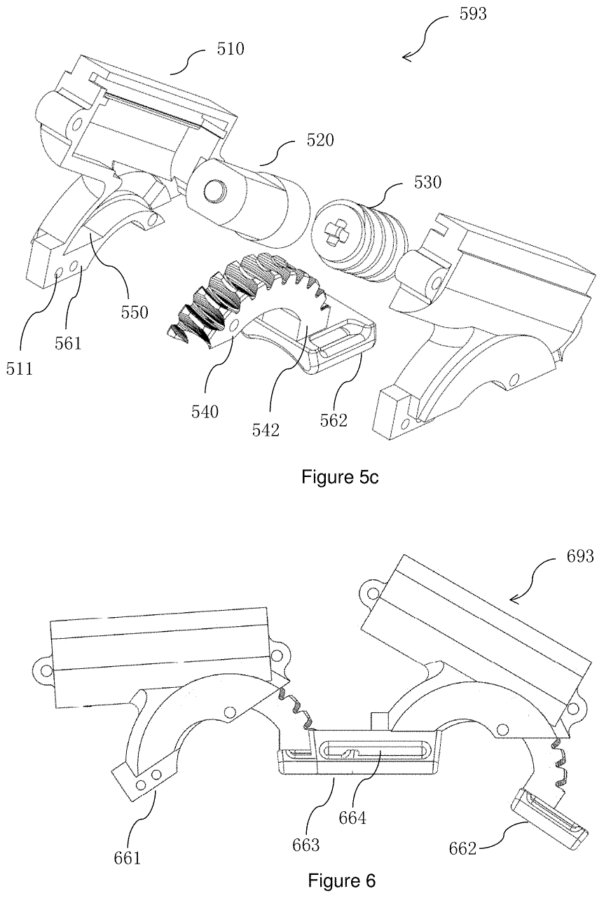

Now referring to FIG. 6, a perspective side view of a further example driving assembly for moving associated body parts according to a preferred embodiment of the present disclosure is illustrated, according to which two manipulators 693 are cascaded for assisting movement of or control over a body part, such as a finger, wherein the tail end of the leftmost connector 661 of the left manipulator is configured to be mountable at a first body part or a palm plate, while its head end and the tail end of the right manipulator are connected or coupled in a manner resembling to the same illustrated in FIGS. 3a via an intermediate connector 663 with a prolonged length/dimension. The intermediate connector 663 comprises a first and a second coupling members spaced apart in a length direction and arranged at opposing ends for coupling with respective ends of the left and right manipulators or a free end of a partial gear wheel thereof; and an opening 664, preferably a through slot, extended along a width direction adapted for insertion of a fastening member such as a band/rope for winding on a second body part (or a finger) for positioning/mounting the driving assembly properly over a finger or a corresponding finger joint (not shown). The dimension or the length and/or the width of the intermediate connector might be adapted to a predetermined dimension of the body part to provide or form an optimized joint with a desirable stiffness between the left and right manipulators for enabling/facilitating optimized operations thereof; and it might also differs from the same of the leftmost connector 661 arranged at the tail end of the left manipulator and/or the rightmost connector 662 arranged at the head end of the right manipulator, as illustrated in the figure. In some embodiments, the left and right manipulators are integrated with the intermediate and/or the rightmost connectors.

The present disclosure is described according to specific embodiments, but those skilled in the art will appreciate that various changes and equivalents might be made without departing from the scope of the present disclosure. In addition, many modifications might be made to the present disclosure without departing from the scope of the invention in order to adapt to specific circumstances or components of the present disclosure. Accordingly, the present disclosure is not limited to the specific embodiments disclosed herein, and shall include all embodiments falling within the scope of the claims.

1. A driving assembly for moving or rotating, or assisting or resisting movement of, a first body part relative to a second body part and preferably enabling flexion, extension, pronation, supination, rotation, abduction, and adduction of the first body part relative to the second body part, comprising at least one manipulator unit for body part including a housing for accommodating at least partly of an actuator, a torque/force transmission gear, a partial gear wheel operatively connected with each other and a first connector arranged at a first/tail end of the manipulator unit for coupling with the first body part and a second connector movably arranged at a second/head end of the manipulator unit for coupling with the second body part and preferably further coupled with and driven by the partial gear wheel actuated by the actuator via the torque transmission gear for coupling and moving with the second body part, and preferably an arc channel or rail for confining and/or guiding reciprocal or back and forth movement and preferably rotational movement of the partial gear wheel;
wherein the second connector is configured to be operated interchangeably between an idle/inactive state in which the second connector is positioned at or adjacent to the second end and an active state in which the second connector is driven to move away from or move toward the second end along a circumferential direction by the partial gear wheel or to rotate a predetermined or desired first angle about a rotation axis defined by a curvature of the partial gear wheel and preferably the arc channel or rail in response to a preset instruction or a received real-time instruction, so as to drive the second body part coupled therewith to rotate/pivot a predetermined or desired second angle about a body joint positioned between the first and the second body parts.

2. The driving assembly according to claim 1, comprising a plurality of manipulator units of identical dimension or of different dimensions arranged in serial and/or parallel connection with each other; wherein respective first connectors of the plurality of manipulator units are configured to couple with each other and/or with same first body part and respective second connectors of the plurality of manipulator units are configured to couple with each other and/or with same second body part for providing extra degrees of freedom of rotation or motion for the second body part in relation to the first body part; and/or
wherein the second connector of one of the plurality of manipulator units is configured to couple with the first connector of other one of the plurality of manipulator units, so as to provide additional rotation angle and/or extra degrees of freedom of motion for one or more body parts or joints coupled with and driven singly/respectively or collectively by one or more of the plurality of manipulator units.

3. The driving assembly according to claim 1, further comprising one or more stoppers arranged at, and preferably adjacent to a tail end of, the partial gear wheel, the arc channel or rail, and/or the housing for confining movement and/or providing safety limits for movement of the partial gear wheel operated in both idle and active states.

4. The driving assembly according to claim 1, wherein the actuator is selected from a group comprising a DC motor, an AC/DC motor, a hydraulic motor, a pneumatic motor, a brush/brushless motor, a piezo motor, a memory shaped alloy actuated by AC/DC electricity, batteries, chemicals, change of temperatures, change of pressures, lights or electromagnetism, sound or energy waves; and/or
wherein the torque transmission gear is selected from a group comprising a worm gear, a spur gear, a bevel gear, a belt gear, a pulley gear, and a combination thereof; and/or
wherein the partial gear wheel is selected from a group comprising a worm wheel, a spur wheel, a bevel wheel, a belt wheel, a pulley wheel, and a combination thereof; and/or
wherein the partial gear wheel is a wheel with less than 360 degrees, preferably less than 135 degrees, but more than 0 degree of rotation.

5. The driving assembly according to claim 1, wherein the manipulator unit further comprises one or more sensors and/or one or more controllers arranged at or in the housing to measure or control/process data related to positions, angles of rotation, degrees of movement, or orientations of the second connector, forces or pressures exerted or received by the second connector, ambient sound, light, and/or temperature;
wherein measured and output or feedback data generated by the sensors and/or the controllers are communicated or transmitted, preferably to a remote/external terminal, via one or more of wired and/or wireless technologies and protocols including Bluetooth, Wifi, Infrared, Zigbee, GSM, LTE, and a combination thereof.

6. The driving assembly according to claim 1, wherein an adjacent pair of a first and a second manipulator units are coupled with each other via an interconnection of the second connector of the first manipulator unit and the first connector of the second manipulator unit, an interconnection of the first connector of the first manipulator unit and the second connector of the second manipulator unit, an interconnection of the first connector of the first manipulator unit and the first connector of the second manipulator unit, or an interconnection of the second connector of the first manipulator unit and the second connector of the second manipulator unit.

7. A robotic hand or glove for moving or rotating, or assisting or resisting movement of, a human hand and/or its fingers comprising
a driving assembly for moving or rotating, or assisting or resisting movement of, a first body part relative to a second body part and preferably enabling flexion, extension, pronation, supination, rotation, abduction, and adduction of the first body part relative to the second body part, comprising at least one manipulator unit for body part including a housing for accommodating at least partly of an actuator, a torque/force transmission gear, a partial gear wheel operatively connected with each other and a first connector arranged at a first/tail end of the manipulator unit for coupling with the first body part and a second connector movably arranged at a second/head end of the manipulator unit for coupling with the second body part and preferably further coupled with and driven by the partial gear wheel actuated by the actuator via the torque transmission gear for coupling and moving with the second body part, and preferably an arc channel or rail for confining and/or guiding reciprocal or back and forth movement and preferably rotational movement of the partial gear wheel;
wherein the second connector is configured to be operated interchangeably between an idle/inactive state in which the second connector is positioned at or adjacent to the second end and an active state in which the second connector is driven to move away from or move toward the second end along a circumferential direction by the partial gear wheel or to rotate a predetermined or desired first angle about a rotation axis defined by a curvature of the partial gear wheel and preferably the arc channel or rail in response to a preset instruction or a received real-time instruction, so as to drive the second body part coupled therewith to rotate/pivot a predetermined or desired second angle about a body joint positioned between the first and the second body parts;
further comprising a palm plate for mounting the robotic hand or glove on the human hand wrist, and/or a forearm and for mounting a plurality of manipulator units for use with and manipulation of a plurality of fingers of the human hand, wherein each of the plurality fingers is coupled with and actuated by one or more manipulator units configured for and capable of providing one or more degrees of freedom; and
one or more sensors or sensing devices including an electromyography sensor for measuring electrical activity of muscles of the human hand and fingers, a force sensitive resistor for measuring or gathering information about pressure at each fingertip of the human hand, an accelerometer for measuring acceleration forces of the human hand and fingers, and/or a gyroscope for measuring or maintaining orientation and angular velocity of the human hand and fingers;
a micro-controller configured to control/manipulate, coordinate, and/or synchronize movement of the plurality of manipulator units in response to data measured and provided by the one or more sensors for effecting desired operations of the robotic hand for assisting or resisting movement of the human hand.

8. The robotic hand or glove according to claim 7, wherein the palm plate further comprises at least one first attachment connector having a first engagement member arranged at or in proximity of a proximal end of the palm plate for coupling with a human wrist and/or forearm and/or at least one second attachment connector having a second engagement member arranged at or in proximity of a distal end of the palm plate for coupling with the first connector of a proximal one of the plurality of manipulator units.

9. The robotic hand or glove according to claim 7, wherein two manipulator units arranged in a cascaded manner are coupling with one of the fingers or thumb of the human hand to provide 2 degrees of freedom and actuate rotation of one or more finger joints including PIP joint and/or MCP joint to provide flexion and/or extension of the finger or thumb; and/or two or three manipulator units arranged in a cascaded manner are coupling with other one of the fingers, including index finger, middle finger, ring finger, and little finger, of the human hand to provide 2 or 3 degrees of freedom and actuate rotation of one or more finger joints including PIP joint, MCP joint, and/or DIP joint; or thumb of the human hand to provide 2 or 3 degrees of freedom and actuate rotation of one or more finger joints including IP joint, MCP joint, and CMC joint; and/or wherein extra one or more manipulators arranged in standalone or combination with others are coupling with the thumb and other fingers to provide extra degree of freedom for abduction and/or adduction.

10. The robotic hand or glove according to claim 7, wherein each of manipulator units is configured to provide itself or a segment of an associated finger of the human hand with rotation movement preferably greater than 0 degree and more preferably less than 180 degree.

11. A driving assembly for moving or rotating, or assisting or resisting movement of, a first body part relative to a second body part and preferably enabling flexion, extension, pronation, supination, rotation, abduction, and adduction of the first body part relative to the second body part, comprising at least one manipulator unit for body part including a first connector arranged at a first/tail end of the manipulator unit for coupling with the first body part and a second connector movably arranged at a second/head end of the manipulator unit for coupling and moving with the second body part, and a curved driving member positioned between the first end and the second end, wherein the second connector is actuated and driven by the curved driving member for effecting reciprocal or back and forth movement and preferably rotational movement along a curved path between the first connector and the second connector;
wherein the second connector is configured to be operated interchangeably between an idle/inactive state in which the second connector is positioned at or adjacent to the second end and an active state in which the second connector is driven to move away from or move toward the second end along a circumferential direction by the curved driving member or to rotate a predetermined or desired first angle about a first rotation axis defined by a curvature of the curved driving member in response to a preset instruction or a received real-time instruction, so as to drive the second body part coupled therewith to rotate/pivot a predetermined or desired second angle about a body joint/a second rotation axis positioned between the first and the second body parts; and preferably, the first rotation axis is substantially parallel to or coinciding with the second rotation axis for optimizing a driving efficiency thereof.

12. The driving assembly according to claim 11, wherein the curved driving member comprises a housing for accommodating at least partly of an actuator, a torque/force transmission gear, a partial gear wheel operatively connected with each other; wherein the second connector is connected with and driven by the partial gear wheel actuated by the actuator via the torque transmission gear, so as to be moved interchangeably between a first angular position and a second angular position along the circumferential direction in response to the preset instruction or the received real-time instruction.
